(19) United States
(12) Patent Application Publication
(10) Pub. No.: US 2014/0188056 A1
(43) Pub. Date: Jul. 3, 2014

(54) COMPOSITIONS AND METHODS FOR TRANSDERMAL DELIVERY OF NON-STEROIDAL ANTI-INFLAMMATORY AGENTS

(71) Applicant: Noven Pharmaceuticals, Inc., Miami, FL (US)

(72) Inventors: Keita Mori, Miami, FL (US); Puchun LlU, Miami, FL (US)

(73) Assignee: Noven Pharmaceuticals, Inc., Miami, FL (US)

(21) Appl. No.: 14/141,946

(22) Filed: Dec. 27, 2013

Related U.S. Application Data
(60) Provisional application No. 61/747,039, filed on Dec. 28, 2012.

(51) Int. Cl. A61K 9/70; A61K 31/192; A61K 47/32

(52) U.S. Cl. CPC A61K 9/703; A61K 47/32; A61K 31/192
USPC 604/290; 514/772.6; 514/772.1; 514/570; 604/307; 424/449

(57) ABSTRACT

Compositions for the transdermal delivery of NSAIDs in a flexible, finite form are described. The compositions comprise a polymer matrix that includes an NSAID and a polymer matrix comprising a blend of silicone and acrylic polymers. A flexible, occlusive backing material also is disclosed.

### CROSS-REFERENCE TO RELATED APPLICATIONS

This application claims the benefit under 35 USC §119(e) to U.S. provisional application 61/747,039, filed Dec. 28, 2012, the entire contents of which are incorporated herein by reference in their entirety.

### BACKGROUND

The present invention relates generally to the transdermal delivery of non-steroidal anti-inflammatory agents (NSAIDs), and to compositions and methods for transdermally delivering NSAIDs, such as may be desired for treating or reducing pain and/or inflammation. The present invention also relates to a flexible, occlusive backing material suitable for use with any flexible, finite transdermal drug delivery system.

When NSAIDS are administered orally, they may have various adverse effects, such as gastrointestinal tract disturbances, ulcers, and bleeding. Moreover, when an NSAID is administered to treat local pain, oral administration may lead to unnecessary and/or unwanted systemic effects. Transdermal delivery offers the possibility of treating local pain while avoid the adverse effects associated with oral administration.

Many factors influence the design and performance of transdermal drug delivery compositions. These include the individual drugs themselves, the physical and chemical characteristics of the compositions' components and their performance and behavior relative to other components, external and environmental conditions during manufacturing and storage, properties of the application site, the desired rate of drug delivery and therapeutic onset, the desired drug delivery profile, and the intended duration of delivery, among others.

Compositions for the transdermal delivery of NSAIDs are known, but there remains a need for compositions that exhibit suitable physical and pharmacokinetic properties.

### SUMMARY

Described are compositions for the transdermal delivery of an NSAID in the form of a flexible finite system for topical application, comprising a polymer matrix comprising (i) a therapeutically effective amount of an NSAID, such as flurbiprofen; (ii) a silicone polymer; and (iii) an acrylic polymer or an acrylic block copolymer. In some embodiments, the polymer matrix further comprises a styrene-isoprene-styrene block copolymer. In some embodiments, the polymer matrix further comprises a penetration enhancer.

In some embodiments, the polymer matrix comprises a non-functional acrylic polymer. In some embodiments, the polymer matrix comprises an acrylic block copolymer.

In some embodiments, the polymer matrix comprises about 4 to 5% w/w acrylic polymer, and/or about 5% w/w acrylic block copolymer, and/or about 1% w/w styrene-isoprene-styrene block copolymer and/or at least about 80% w/w silicone polymer.

In some embodiments, the system achieves delivery of the NSAID over a period of time of at least 8 hours, or over a period of time of at least 12 hours, or over a period of time of at least 24 hours.

In some embodiments, the composition further comprising a backing layer, such as a flexible, occlusive backing layer, optionally a flexible, occlusive and stretchable backing layer. In some embodiments, the flexible, occlusive backing layer is comprised of a fabric backing material coated with an occlusive coating, such as a polyisobutylene coating.

Also described are methods for the transdermal delivery of an NSAID, comprising topically applying a composition as described herein to the skin or mucosa of a subject in need thereof.

Also described are flexible, finite systems for the transdermal delivery of an active agent comprising (i) a polymer matrix comprising the active agent and one or more polymers and (ii) a flexible, occlusive backing layer comprised of a fabric backing material coated with an occlusive coating. In some embodiments, the flexible, occlusive backing layer is stretchable. In some embodiments, the flexible, finite system is stretchable.

### BRIEF DESCRIPTION OF THE DRAWINGS

FIG. 1 shows the results of in vitro drug flux studies of flubiprofen from a system comprising a polymer matrix as described herein comprising a silicone polymer and an acrylic polymer and different backing layers (♦—PIB coated cloth backing; ▪—polyolefin/cloth laminated film backing; ▴—Scotchpack® 9732; [symbol] —Yakuban Tape commercial flubiprofen patch (lowest line)).

FIGS. 2A-E show the results of in vitro drug flux studies of flubiprofen from a system comprising a polymer matrix as described herein comprising a silicone polymer, an acrylic polymer, and a styrene-isoprene-styrene block copolymer compared to commercial products (♦—composition as described herein; or ▪—Yakuban Tape commercial flubiprofen patch; ▴—Mohrus Tape commercial ketoprofen patch).

FIGS. 3A-D show the results of in vitro drug flux studies of flubiprofen from systems comprising a polymer matrix as described herein comprising a silicone polymer and one of two different acrylic block copolymers (ABCs) compared to commercial products (♦—Formula 4-1; ▪—Formula 4-2; or [symbol] —Yakuban Tape commercial flubiprofen patch; ▴—Mohrus Tape commercial ketoprofen patch).

FIG. 4A-F shows the drug flux from polymer matrices prepared from compositions described herein (formulation 5-1) as compared to the flux from a commercial product, Yakuban Tape (a flurbiprofen commercial patch).

FIG. 5A-E shows the drug flux from polymer matrices prepared from compositions described herein (formulation 5-2) as compared to the flux from a commercial product, Yakuban Tape (a flurbiprofen commercial patch).

FIG. 6 shows the results of an assessment of the compositions described herein in the yeast-induced hyperalgesia animal (rat) model for pain as compared to commercial Yakuban Tape and Mohrus Tape.

### DETAILED DESCRIPTION

Described herein are compositions and methods for the transdermal delivery of an NSAID in a flexible, finite form (e.g., “patch”-type systems). The compositions in flexible, finite form comprise a polymer matrix that includes an NSAID or a pharmaceutically acceptable salt thereof and a backing layer. The compositions exhibit satisfactory physical properties while also achieving satisfactory pharmacokinetic profiles. In specific embodiments, the polymer matrix comprises (i) a therapeutically effective amount of an NSAID; (ii) a silicone polymer; and (iii) an acrylic polymer or an acrylic block copolymer. In further specific embodiments, the polymer matrix further comprises a styrene-isoprene-styrene block copolymer. In accordance with any embodiments, the polymer matrix may further comprise a penetration enhancer.

### Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein means that the described composition (e.g., polymer matrix, etc.) comprises less than about 5%, less than about 3%, or less than about 1% by weight, based on the total weight of the composition at issue, of the excluded component(s).

As used herein “subject” denotes any mammal in need of drug therapy, including humans. For example, a subject may be suffering from or at risk of developing a condition that can be treated or prevented with an NSAID (such as pain or inflammation), or may be taking an NSAID for other purposes.

As used herein, the terms “topical” and “topically” mean application to a skin or mucosal surface of a mammal, while the terms “transdermal” and “transdermal” connote passage through the skin or mucosa (including oral, buccal, nasal, rectal and vaginal mucosa), into systemic circulation. Thus, the compositions described herein may be applied topically to a subject to achieve transdermal delivery of an NSAID.

As used herein, the phrases “therapeutically effective amount” and “therapeutic level” mean that drug dosage or plasma concentration in a subject, respectively, that provides the specific pharmacological effect for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

The compositions described herein are in a “flexible, finite form.” As used herein, the phrase “flexible, finite form” means a substantially solid form capable of conforming to a surface with which it comes into contact, and capable of maintaining contact so as to facilitate topical application. Such systems in general are known in the art and commercially available, such as transdermal drug delivery patches.

The compositions comprise a drug-containing polymer matrix that releases the NSAID upon application to the skin (or any other surface noted above). The compositions in flexible, finite form also include a backing layer in addition to the drug-containing polymer matrix layer. In some embodiments, the compositions in flexible, finite form may include a release liner layer in addition to a drug-containing polymer matrix layer and backing layer.

As used herein, “drug-containing polymer matrix” refers to a polymer composition which contains one or more drugs, such as one or more NSAIDs, and a polymer, such as a pressure-sensitive adhesive polymer or a bioadhesive polymer. A polymer is an “adhesive” or “bioadhesive” if it has the properties of adhesiveness per se. Other polymers can function as an adhesive or bioadhesive by the addition of tackifiers, plasticizers, crosslinking agents or other excipients. Thus, in some embodiments, the polymer optionally comprises tackifiers, plasticizers, crosslinking agents or other additives known in the art.

As used herein, the term “pressure-sensitive adhesive” refers to a viscoelastic material which adheres instantaneously to most substrates with the application of very slight pressure and remains permanently tacky. As noted above, a polymer is a pressure-sensitive adhesive polymer if it has the properties of a pressure-sensitive adhesive per se. Other polymers may function as a pressure-sensitive adhesive by admixture with tackifiers, plasticizers or other additives. The term pressure-sensitive adhesive also includes mixtures of different polymers.

In some embodiments, the polymer matrix is a pressure-sensitive adhesive at room temperature and exhibits desirable physical properties, such as good adherence to skin, ability to be peeled or otherwise removed without substantial trauma to the skin, retention of tack with aging, etc. In some embodiments, the polymer matrix has a glass transition temperature (T_{g}), measured using a differential scanning calorimeter, of between about −70° C. and 0° C.

In some embodiments, the compositions in flexible, finite form are “monolithic” or “monolayer” systems, such that the drug-containing polymer matrix layer is the only polymeric layer present other than the backing layer and the release liner, if present. In such embodiments, the polymer matrix functions as both the drug carrier and the means of affixing the system to the skin or mucosa.

Polymer Matrix

In accordance with some embodiments, the compositions described herein comprise a polymer matrix that comprises, consists essentially of, or consists of, an NSAID and/or pharmaceutically acceptable salt(s) thereof and a silicone polymer, an acrylic polymer and/or an acrylic block copolymer and, optionally, a styrene-isoprene-styrene block copolymer. In this context, the phrase “consists essentially of” means that the polymer matrix is substantially free of other polymer components (e.g. substantially free of polymers other than silicone polymer(s), acrylic polymer(s), and styrene-isoprene-styrene block copolymer(s) and skin penetration enhancers, although it may include other excipients known to be useful in transdermal compositions (such as tackifiers, plasticizers, crosslinking agents or other excipients known in the art) as long as those other excipients do not degrade the physical and/or pharmacokinetic properties of the compositions to pharmaceutically unacceptable levels. In accordance with some embodiments, the compositions described herein comprise a polymer matrix that comprises, consists essentially of, or consists of, an NSAID and/or pharmaceutically acceptable salt(s) thereof a silicone polymer, an acrylic polymer and/or an acrylic block copolymer and, optionally, a styrene-isoprene-styrene block copolymer and, optionally, one or more skin penetration enhancers.

NSAID

NSAIDs are known in the art and include ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, niflumic acid, aspirin, diflunisal, and salsalate.

In specific embodiments, the NSAID is flurbiprofen. Flurbiprofen has anti-inflammatory, analgesic and antipyretic properties. It is used, for example, to treat rheumatoid arthritis, osteoarthritis, and to prevent miosis during ocular surgery.

The compositions described herein may be formulated with an NSAID in its free acid form, or as any pharmaceutically acceptable ester thereof, or any combinations thereof. Exemplary suitable pharmaceutically acceptable salts are salts of weak inorganic and organic acids, and quaternary ammonium salts. These include without limitation, salts with acids such as sulfuric, phosphoric, hydrochloric, hydrobromic, hydriodic, sulfamic, citric, lactic, maleic, malic, succinic, tartaric, cinnamic, acetic, benzoic, gluconic, or ascorbic acid, or quaternary ammonium salts with organic esters of sulfuric, hydrohalic, or aromatic sulfonic acids, such as methyl chloride, methyl bromide, ethyl chloride, propyl chloride, butyl chloride, isobutyl chloride, benzylchloride, benzyl bromide, phenethyl bromide, naphthymethyl chloride, dimethyl sulfate, methyl benzenesulfonate, ethyl toluenesulfonate, ethylene chlorohydrin, propylene chlorobydrin, allyl bromide, methylallyl bromide or crotyl bromide esters.

The compositions described herein include a therapeutically effective amount of NSAID or pharmaceutically acceptable salt(s) thereof. Generally, the amount of NSAID is from about 0.1% to about 50%, including from about 1% to about 20%, such as from about 1% to about 10% by weight, such as about 1, about 2, about 3, about 4, about 5, about 6, about 7, about 8, about 9 or about 10% by weight, based on the total dry weight of the polymer matrix. In specific embodiments, the polymer matrix comprises about 3-5% by weight NSAID, based on the total dry weight of the polymer matrix, such as about 3% or about 5% by weight NSAID, based on the total dry weight of the polymer matrix.

When the compositions are used for local effect, they may include from about 20 to about 35 mg of NSAID (such as flurbiprofen). The compositions have specific advantages when used for local effect, e.g., to treat conditions at or near the application site. In addition to avoiding the gastrointestinal tract and associated side effects, the compositions are able to deliver a high dose of NSAID directly to the site to be treated, while reducing or minimizing undesired systemic effects.

### Silicone Polymers

As noted above, in some embodiments the polymer matrix comprises one or more silicone polymers, such as one or more pressure-sensitive adhesive silicone polymers. Silicone polymers suitable for use in polymer matrix compositions are known.

The term “silicone-based” polymer is used interchangeably with the terms silicon polymers, siloxane, polysiloxane, and silicones as used herein and as known in the art. A suitable silicone-based polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives.

Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989).

Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4200 series, such as -4202 and -42-3, and the -4500 series, such as -4502, such as -4503, and -4600 series.

Further details and examples of silicone pressure-sensitive adhesives which are useful in the polymer matrices and compositions and methods described herein are mentioned in the following U.S. Pat. Nos. 4,591,622; 4,584,355; 4,585,836; and 4,655,767, which are all expressly incorporated by reference herein in their entireties. It should also be understood that silicone fluids are also contemplated for use in the polymer matrices and methods described herein.

### Acrylic Polymers

As noted above, in some embodiments the polymer matrix comprises one or more acrylic polymers, such as one or more pressure-sensitive adhesive acrylic polymers. Acrylic polymers suitable for use in polymer matrix compositions are known.

The term “acrylic polymer” is used here as in the art interchangeably with “polyacrylate,” “polyacrylic polymer,” and “acrylic adhesive.” The acrylic-based polymers can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives.

The acrylic polymer can include copolymers, terpolymers and multipolymers. For example, the acrylic polymer can be any of the homopolymers, copolymers, terpolymers, and the like of various acrylic acids. In some embodiments, the acrylic polymer constitutes from about 2% to about 95% by weight of the polymer content of the polymer matrix, including about 3% to about 90% and about 5% to about 85%, such as 2% to 95%, 3% to 90% and 5% to 85%. In some embodiments, the amount and type of acrylic polymer is dependent on the type and amount of therapeutically active agents used.

Acrylic polymers useful in practicing the invention include polymers of one or more monomers of acrylic acids and other copolymerizable monomers. The acrylic polymers also include copolymers of alkyl acrylates and/or methacrylates and/or copolymerizable secondary monomers or monomers with functional groups. Combinations of acrylic-based polymers based on their functional groups is also contemplated. Acrylic-based polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups. In other embodiments, the acrylic polymer does not include hydroxy functional groups.

In accordance with specific embodiments, the polymer matrix comprises or consists of one or more non acid-functional acrylic polymers as the polymer component. Non acid-functional acrylic polymers include those formed from acrylic esters copolymerized with other monomers that do not include acid-functional groups. Non acid-functional acrylic polymers include homopolymers, copolymers, terpolymers, etc., of acrylic acids and esters. As used herein, “non acid-functional acrylic polymer” includes polymers that include monomers that have one or more amide groups. In specific embodiments, the non acid-functional acrylic polymer includes methacrylate monomers and 2-ethylhexyl acrylate monomers. In specific embodiments the non acid-functional acrylic polymer includes methacrylate monomers, 2-ethylhexyl acrylate monomers, and amide-group containing monomers.

In some embodiments, the acrylic polymer component of the polymer matrix consists of a single acrylic polymer. In other embodiments, the acrylic polymer component of the polymer matrix comprises a blend of a first acrylic polymer and a second acrylic polymer, and optionally includes additional (e.g., a third or more) acrylic polymers.

When the acrylic polymer component includes more than one acrylic polymer, the polymers can be present in any ratio that results in a product with satisfactory physical and pharmacokinetic properties. For example, the acrylic polymer component can include from 0-100% of a first acrylic polymer and from 100-0% of a second acrylic polymer, based on the total dry weight of the acrylic component, including about 10 to about 90%, about 15-about 85%, about 20 to about 80%, about 25 to about 75%, about 33 to about 66%, and about 50% of the first acrylic polymer, and the balance being the second (or third, etc.) acrylic polymer(s). In specific embodiments, the acrylic polymer component includes about 80% of a first acrylic polymer and about 20% of a second acrylic polymer, based on the total polymer content.

Suitable acrylic polymers which are commercially available include those sold by Henkel (Dusseldorf, Germany), under the Duro-Tak® brand such as Duro-Tak 900A or Duro-Tak 87-9900, and those sold by Monsanto (St. Louis, Mo.) under Gelva® Multipolymer Solution brand, such as Gelva 3087 and Gelva-3235. Other suitable acrylic polymers are known in the art. See, e.g., the non acid-functional acrylic polymers described in Satas, “Acrylic Adhesives, HANDBOOK OF PRESSURE-SENSITIVE ADHESIVE TECHNOLOGY, 2nd ed., pp. 396-456 (D. Satas, ed.), Van Nostrand Reinhold, N. Y. (1989); “Acrylic and Methacrylic Ester Polymers,” POLYMER SCIENCE AND ENGINEERING, Vol. 1, 2nd ed., pp 234-268, John Wiley & Sons, (1984).

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.). Van Nostrand Reinhold, New York (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

### Acrylic Block Copolymers

As noted above, in some embodiments the polymer matrix comprises one or more acrylic block copolymers, such as one or more pressure-sensitive adhesive acrylic block copolymers.

In accordance with some embodiments, the polymer matrix comprises an acrylic block copolymer that is a conjugate of a non-functional acrylic pressure-sensitive adhesive (such as any described above) and silicone fluid polydimethylsiloxane (PDMS). Such a block copolymer may be made by the following reaction scheme:

MA+2-EHA+CH₂=CH-PDMS-OSiMe₃→[(MA)x−(2-EHA)y]p-[CH2-CH-PDMS]q

(conjugates of MA/2-EHA copolymer and PDMS)

where:
p=50%, q=50%
x=50%, y=50% or x=80%, y=20% or x=100%, y=0%
MA=methyl acrylate=CH₂═CH—COOCH₃
2-EHA=2-ethylhexyl acrylate=CH₂═CH—COO—CH₂CH(CH₂CH₃)(CH₂)₃CH₃
PDMS with a double bond, preferably OH free as indicated above, can be CH₂═CH-PDMS, or CH₂═CH-alkylene-PDMS, or CH₂═CH—O-PDMS, or CH₂═CH—COO-alkylene-PDMS, where alkylene is an alkylene group.

In accordance with some embodiments, the polymer matrix comprises an acrylic block copolymer that is a non-functional acrylic pressure-sensitive adhesive (such as any described above) with trimethylsiloxysilane moieties. Such a block copolymer may be made by the following reaction scheme:

MA+2-EHA+APTMS→[(MA)x−(2-EHA)y]p-[APTMS]q

(MA/2-EHA copolymers with APTMS)

where:
p=50%, q=50%
x=50%, y=50% or x=80%, y=20% or x=100%, y=0%
APTMS=3-acryloxypropyl tri(trimethylsiloxy)silane=CH₂═CH—COO—(CH₂)₃—Si(O—Si(CH₃)₃)₃

Suitable acrylic block copolymers are available commercially, such as from Henkel (e.g., Henkel 14700-14 or Duro-Tak 87-9900).

### Other Polymers

As noted above, in some embodiments the polymer matrix comprises one or more rubber-based polymers, such as one or more rubber-based pressure-sensitive adhesives, such as natural or synthetic polyisoprene, polybutylene, polyisobutylene, styrene-butadiene polymers, styrene-isoprene-styrene block copolymers, hydrocarbon polymers, such as butyl rubber, halogen-containing polymers, such as polyacrylic-nitrile, polytetrafluoroethylene, polyvinylchloride, polyvinylidene chloride, and polychlorodiene, and other copolymers thereof. In specific embodiments, the polymer matrix comprises one or more styrene-isoprene-styrene block copolymers. Such polymers suitable for use in polymer matrix compositions are known and are available commercially, such as Kraton® D111 KT.

As noted above, in some embodiments, the polymer matrices of the compositions described herein consist essentially of the NSAID or pharmaceutically acceptable salt(s) thereof and one or more of the polymer(s) described above, although such compositions may include other non-polymer components that do not degrade the physical and/or pharmacokinetic properties of the compositions to pharmaceutically unacceptable levels, such as one or more penetration enhancers, as discussed in more detail below.

### Penetration Enhancers

As noted above, in some embodiments, the polymer matrices of the compositions described herein further comprise one or more penetration enhancers. A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer. In specific embodiments the enhancer(s) serve to both enhance penetration of the NSAID through the stratum corneum and retain the NSAID at a site local to administration.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; glycerol mono-, di- and tri-esters of fatty acids, such as glycerol monooleate; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, a combination of enhancers is used. For example, a dual enhancer system comprising isopropyl myrisate and oleic acid may be particularly useful for formulating NSAIDs, such as flubiprofen.

Generally speaking, the polymer matrices may include NSAID in an amount from about 1% to about 50%, including from about 1% to about 10%, such as from about 1% to about 5%, including about 1%, about 2%, about 3%, about 4% about 5%, about 6%, about 7%, about 8%, about 9%, or about 10% by weight, based on the total dry weight of the polymer matrix, including about 3-5%, about 3% and about 5%.

Generally speaking, the silicone pressure-sensitive adhesive(s), if present, may be present in a range from about 1% to about 99%, including from about 50% to about 99%, such as from about 80% to about 99%, including from about 90% to about 99%, including about 80%, about 81%, about 82% about 83%, about 84%, about 85%, about 86%, about 87%, about 88%, about 89%, about 90%, about 91%, about 92% about 93%, about 94%, about 95%, about 96%, about 97%, about 98%, or about 99%, by weight, based on the total dry weight of the polymer matrix.

Generally speaking, the acrylic polymer(s), if present, may be present in a range from about 1% to about 50%, including from about 1% to about 20%, such as from about 1% to about 10%, including about 2%, about 3%, about 4% about 5%, about 6%, about 7%, about 8%, about 9%, or about 10%, by weight, based on the total dry weight of the polymer matrix.

Generally speaking, the acrylic block copolymer(s), if present, may be present in a range from about 1% to about 50%, including from about 1% to about 20%, such as from about 1% to about 10%, including about 2%, about 3%, about 4% about 5%, about 6%, about 7%, about 8%, about 9%, or about 10%, by weight, based on the total dry weight of the polymer matrix.

Generally speaking, the other polymer(s) (such as, for example, styrene-isoprene-styrene block copolymer(s)), if present, may be present in a range from about 0.1% to about 50%, including from about 0.1% to about 10%, such as from about 0.1% to about 5%, including about 0.2%, about 0.3%, about 0.4%, about 0.5%, about 0.6%, about 0.7%, about 0.8%, about 0.9%, about 1.0%, about 2%, about 3%, about 4%, or about 5%, by weight, based on the total dry weight of the polymer matrix.

Generally speaking, the penetration enhancer(s), if present, each may be present in an amount from about 0.1% to about 10%, such as from about 0.1% to about 5%, including about 0.2%, about 0.4%, about 0.6%, about 0.8%, about 1.0%, about 2%, about 3%, about 4%, about 5%, about 6%, about 7%, about 8%, about 9%, or about 10%, by weight, based on the total dry weight of the polymer matrix. In embodiments using more than one enhancer, each may be present in any amount described herein (e.g., from about 0.1% to about 10%) or the total amount of enhancers may be within the amounts described herein (about 0.1% to about 10%).

While not wanting to be bound by any theory it is believed that the polymer blends described herein balance competing goals and properties of drug solubility and drug delivery. For example, a silicone polymer-based system may have a solubility for the NSAID (such as flurboprofen) that is so low (e.g., 1%) that it is difficult to formulate a sufficient amount of NSAID to achieve delivery over an extended time period. On the other hand, an acrylic polymer-based system may have a solubility for the NSAID (such as flurboprofen) that is so high (e.g., 15%) that very high drug loading is required to achieve drug flux out of the system. The inventors have discovered that the polymer blends described herein, comprising a silicone-based polymer and an acrylic polymer and/or an acrylic block copolymer and, optionally, a styrene-isoprene-styrene block copolymer, balances these competing properties and achieves good drug flux without requiring high drug loading.

Backing Layer

The compositions in flexible, finite form comprise a polymer matrix, such as described above, and a backing layer. The backing layer is impermeable to the drug (e.g., impermeable to the amphetamine) and is adjacent one face of the polymer matrix. The backing layer protects the polymer matrix from the environment and prevents loss of the drug and/or release of other components to the environment during use. In some embodiments, the backing layer is stretchable. In some embodiments, the flexible finite system is stretchable.

In some embodiments, the backing layer is a conventional backing layer as known in the art for transdermal drug delivery systems. For example, in some embodiments the backing layer may be a 3M CoTran 9719 backing layer (a 1.7 mm monolayer of polyethylene with an MVTR of 9.1 g/m2/day) or the like.

In some embodiments, the backing layer is both occlusive and flexible, and, optionally, stretchable, such that it is particularly suitable for use on areas of the body that are flexed and/or experience movement, such as joints, while also providing good drug flux. Such a backing layer can be made, for example, by applying an occlusive coating, such as a coating comprising rubber-based polymers, including polyisobutylene (PIB) and styrene-isoprene-styrene (SIS) block copolymers, to a cloth backing material. A flexible (and, optionally, stretchable), occlusive backing layer exhibits increased flux as compared to conventional non-occlusive stretchablebacking (for example, non-woven fabric), which generally exhibit low drug flux because of their low occlusivity and high moisture vapor transmission rates (MVTR).

The moisture vapor transmission rate of a backing layer as described herein can be controlled, for example, by controlling the thickness of the coating, as illustrated in the examples below. In some embodiments, such a backing layer can be used to manufacture a system with a moisture vapor transmission rate that is the same as or even lower than a comparable system with a plastic backing, as illustrated in the examples below.

Although the flexible (and, optionally, stretchable), occlusive backing layer is discussed and illustrated herein with reference to flexible, finite systems for the transdermal delivery of NSAIDs, it can be used as a backing layer for any flexible, finite transdermal drug delivery system (e.g., for any transdermal drug patch). Indeed, as discussed above, the flexible (and, optionally, stretchable), occlusive backing layer is particularly useful for systems that may be applied to areas of the body that are flexed and/or experience movement, such as joints, while also providing good drug flux, and so may be useful for systems formulated with any active agent.

Release Linter

The compositions in flexible, finite form may further comprise a release liner, typically located adjacent the opposite face of the system as compared to the backing layer. When present, the release liner is removed from the system prior to use to expose the polymer matrix layer prior to topical application. Materials suitable for use as release liners are well-known known in the art and commercially available, such as polyester release liners, including coated polyester release liners.

Methods of Manufacture

The compositions described herein can be prepared by methods known in the art. As one step, the polymer matrices described herein can be prepared by methods known in the art, such as blending (mixing) the polymer components in powder or liquid form with an appropriate amount of drug in the presence of an appropriate solvent, such as a volatile organic solvent, optionally with other excipients. To form a final product, the drug/polymer/solvent mixture may be cast onto a release liner (optionally, at ambient temperature and pressure) followed by evaporation of the volatile solvent(s), for example, at room temperature, slightly elevated temperature, or by a heating/drying step, to form the drug-containing polymer matrix on a release liner. A backing layer may be applied to form a final product.

An exemplary general method for preparing a unit final product of a composition as described herein in a flexible, finite form, is as follows:

1. Appropriate amounts of one or more polymers, solvent(s) and/or co-solvent(s), and optional excipient(s) are combined and thoroughly mixed together in a vessel.

2. The NSAID is added to the mixture and agitation is carried out until the drug is uniformly mixed therein.

3. The composition is transferred to a coating operation where it is coated onto a release liner at a controlled specified thickness. The coated composition is then passed through an oven in order to drive off all volatile processing solvents.

4. The composition coated on the release liner is then brought into contact with a previously prepared laminated backing layer and wound into rolls.

5. Appropriate size and shape delivery systems are die-cut from the roll material and then pouched.

As set forth above, a flexible, occlusive backing layer can be prepared by applying an occlusive coating to a fabric backing material.

The order of steps, the amount of the ingredients, and the amount and time of agitation or mixing may be important process variables which will depend on the specific polymers, active agents, solvents and/or cosolvents, and optional excipients used in the composition, but these factors can be adjusted by those skilled in the art. The order in which each method step is performed can be changed if needed without detracting from the invention.

In accordance with any of the embodiments of compositions described herein, the size of the final product is, in some embodiments, in the range of from about 2 cm² to about 140 cm², including 5 cm², 10 cm², 20 cm², 25 cm², 30 cm², 40 cm², 50 cm², 60 cm², 70 cm², 75 cm², 80 cm², 90 cm², 100 cm², 110 cm², 120 cm², 130 cm², and 140 cm².

Methods of Use

The compositions described herein are useful in methods for the transdermal delivery of an NSAID, including in methods for treating local pain, including chronic or persistent pain, such as may be associated with arthritis, such as rheumatoid arthritis or osteoarthritis. In such embodiments, a composition comprising a therapeutically effective amount of an NSAID, such as flurbiprofen, as described herein is topically applied to a subject in need thereof.

In some embodiments, the compositions achieve transdermal delivery of NSAID over a period of time of at least about 8 hours, including a period of time of at least about 8 hours to at least about 12 hours, at least about 24 hours, or longer.

The compositions described herein achieve a transdermal flux of NSAID (and/or one or more pharmaceutically acceptable salt(s) thereof) that is sufficient to have a therapeutic effect. As used herein, “flux” (also called “permeation rate”) is defined as the absorption of a drug through skin or mucosal tissue, and is described by Fick's first law of diffusion:

J=−D (dCm/dx)

where J is the flux in g/cm²/sec, D is the diffusion coefficient of the drug through the skin or mucosa in cm²/sec and dCm/dx is the concentration gradient of the drug across the skin or mucosa.

The following specific examples are included as illustrative of the compositions described herein. These examples are in no way intended to limit the scope of the invention. Other aspects of the invention will be apparent to those skilled in the art to which the invention pertains.

### EXAMPLE 1

A flexible, occlusive backing layer was prepared by applying a polyisobutylene (PIB) coating to a cloth backing material. The moisture vapor transmission rate (MVTR) of backing layers obtained using different amounts of coating was assessed and compared to the MVTR of Scotchpak® 9732 (3M, St. Paul, Minn.), a polyester film laminate comprised of a polyester layer and an ethylene vinyl acetate copolymer layer.

MVTR was measured by standard procedures, e.g., using cups designated for MVTR evaluation. The cups were loaded with calcium chloride, weighed and then sealed by backing material. The cups were placed in a humid chamber set to 40° C./100% RH. A 24-hour test was run to assess how much moisture passed through the backing material from the humid atmosphere into the cups.

| Sample | MVTR (g/m2/day) |
|---|---|
| Scotchpak 9732 | 27.13 |
| PIB 5 mg/cm²/woven cloth | 19.38 |
| PIB 4 mg/cm²/woven cloth | 42.63 |
| PIB 3 mg/cm²/woven cloth | 302.25 |

### EXAMPLE 2

Drug flux from systems having different backings were assessed.

The following polymer matrix was used for each system:

| | |
|---|---|
| Flurbiprofen: | 3.00% |
| Duro-Tak 900A: | 4.85% |
| Bio-PSA 4502: | 92.15% |

The following backing layers were used:

RN037 72-5: PIB coated cloth backing

RN037 79-7: polyolefin/cloth laminated film backing

RN037 79-8: Scotchpack® 9732

FP-ref: Yakuban Tape (Flurbiprofen commercial patch)

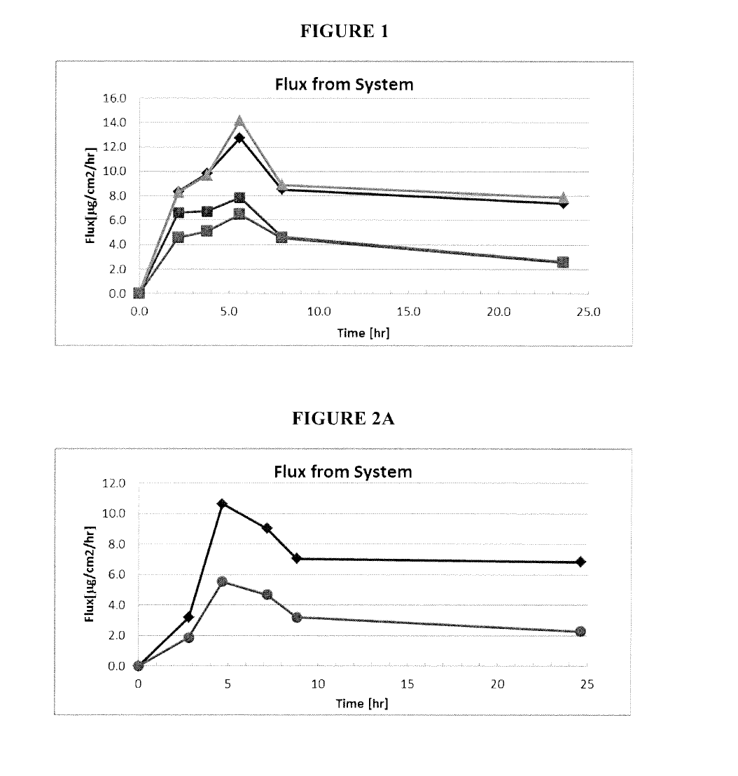

The results are shown in FIG. 1, which shows that the system with the PIB-coated cloth backing achieves a drug flux comparable to that of the system with Scotchpack® 9732 backing layer. (♦—PIB coated cloth backing; ▪—polyolefin/cloth laminated film backing; ▴—Scotchpack® 9732; [symbol] —Yakuban Tape commercial flubiprofen patch (lowest line)).

### EXAMPLE 3

Drug flux from a polymer matrix prepared as follows was assessed and compared with the flux from two commercial products, Yakuban Tape (a flurbiprofen commercial patch) and Mohrus Tape (a ketoprofen commercial patch).

| | |
|---|---|
| Flurbiprofen: | 3.00% |
| Duro-Tak 87-900A: | 4.85% |
| KRATON D1111 KT: | 0.97% |
| Bio-PSA 4503: | 91.18% |

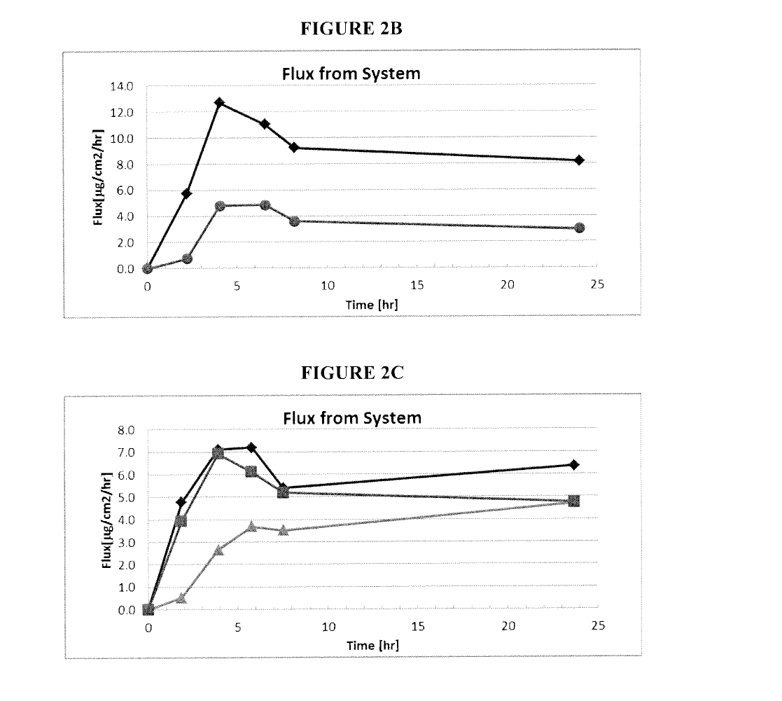

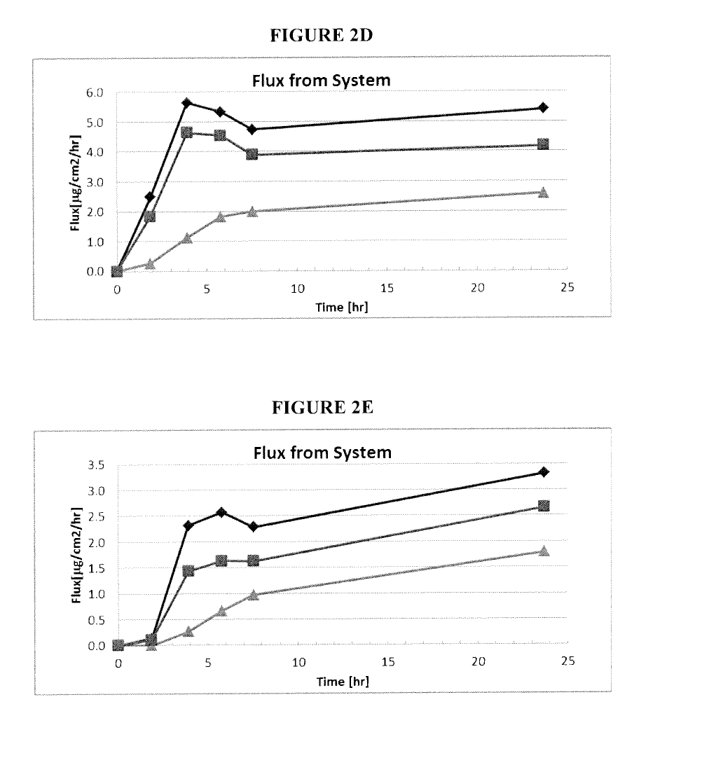

Results for each of five replicate samples are shown in FIGS. 2A-E, and demonstrate that the polymer matrix as described herein achieves a greater, sustained drug flux than the commercial products. (♦—composition as described herein; or ▪—Yakuban Tape commercial flubiprofen patch; ▴—Mohrus Tape commercial ketoprofen patch).

### EXAMPLE 4

Drug flux from polymer matrices prepared using one of two difference acrylic block copolymers (ABCs) was assessed and compared with the flux from two commercial products, Yakuban Tape (a flurbiprofen commercial patch) and Mohrus Tape (a ketoprofen commercial patch).

### Formula 4-1

| | |
|---|---|
| Flurbiprofen: | 3.00% |
| Henkel 14700-14 (ABC) | 4.9% |
| Bio-PSA 4502: | 92.2% |

### Formula 4-2

| | |
|---|---|
| Flurbiprofen: | 3.00% |
| Duro-Tak 87-9900 (ABC) | 4.9% |
| Bio-PSA 4502: | 92.2% |

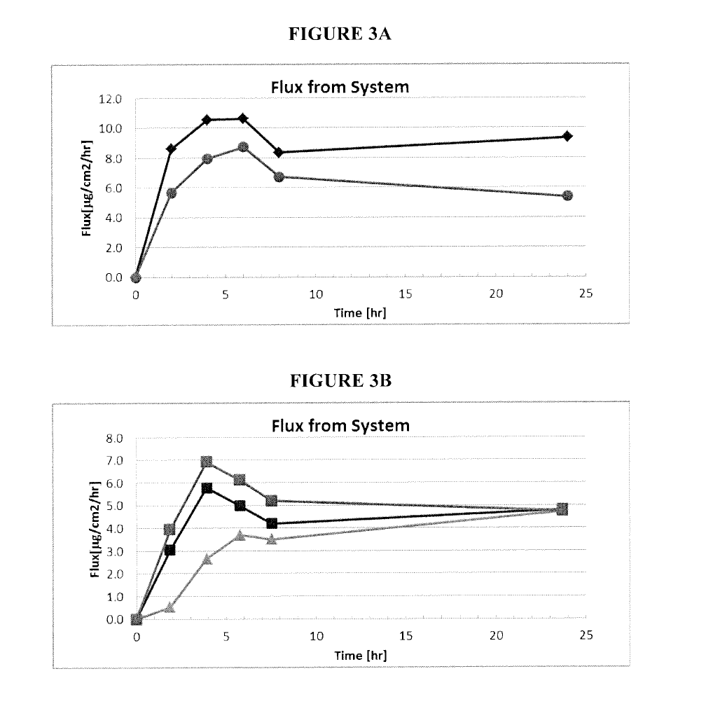

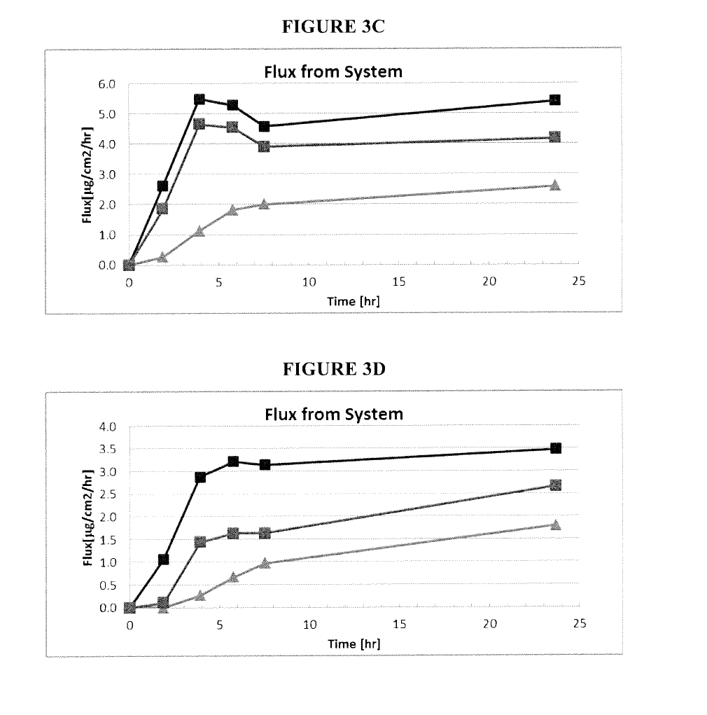

Results are shown in FIGS. 3A-D, and demonstrate that the polymer matrices as described herein achieve a greater, sustained drug flux than the commercial products. (♦—Formula 4-1; ▪—Formula 4-2; or [symbol] —Yakuban Tape commercial flubiprofen patch; ▴—Mohrus Tape commercial ketoprofen patch).

### EXAMPLE 5

Drug flux from polymer matrices prepared from the following formulations was assessed and compared with the flux from a commercial product, Yakuban Tape (a flurbiprofen commercial patch).

### Formula 5-1

| Components | % |
|---|---|
| Flurbiprofen | 5.0 |
| Bio-PSA 4202 | 83.6 |
| Duro-TAK 87-9900 | 4.4 |
| Isopropyl Myristate | 2.0 |
| Oleic Acid | 2.0 |
| Povidone 30 | 3.0 |

### Formula 5-2

| Components | % |
|---|---|
| Flurbiprofen | 5.0 |
| Bio-PSA 4203 | 82.7 |
| Duro-Tak 87-900A | 4.4 |
| SIS D1111 | 0.9 |
| Isopropyl Myristate | 2.0 |
| Oleic Acid | 2.0 |
| Povidone 30 | 3.0 |

The formulations were applied at a coat weight of 10 mg/cm² to Scotchpak 1022 release liner material and provided with a 3M CoTran 9719 backing layer (a 1.7 mm monolayer of polyethylene with an MVTR of 9.1 g/m2/day).

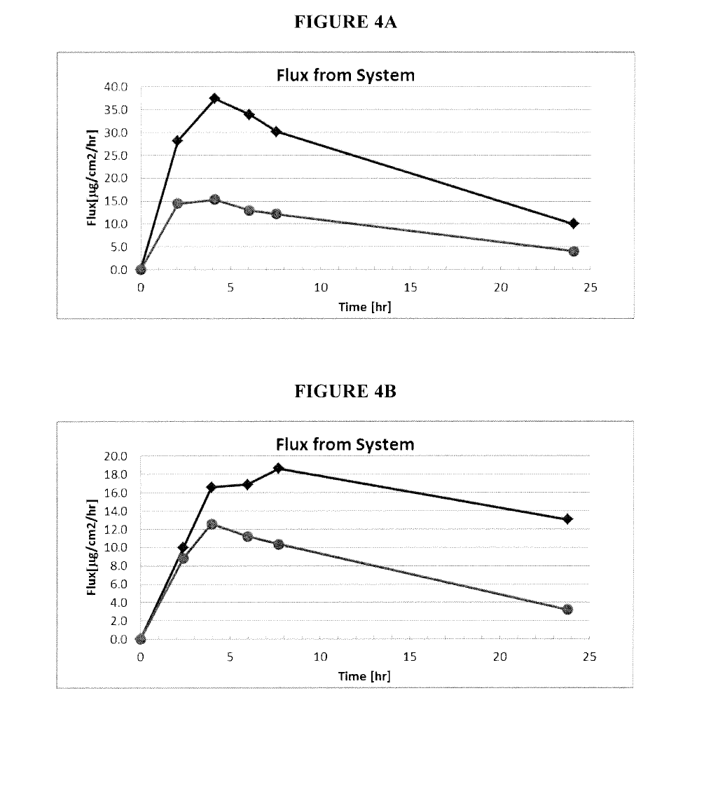

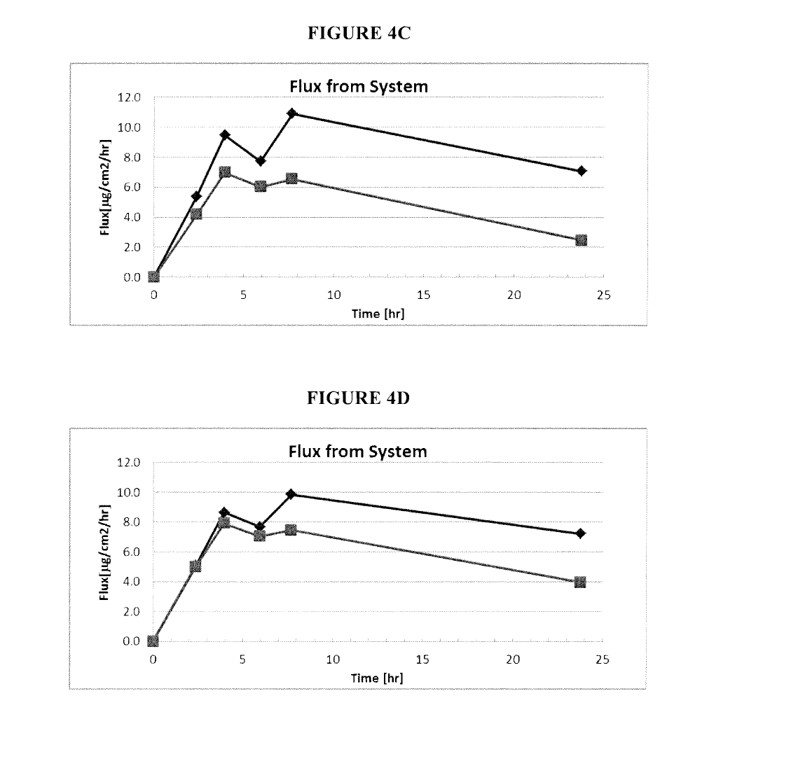

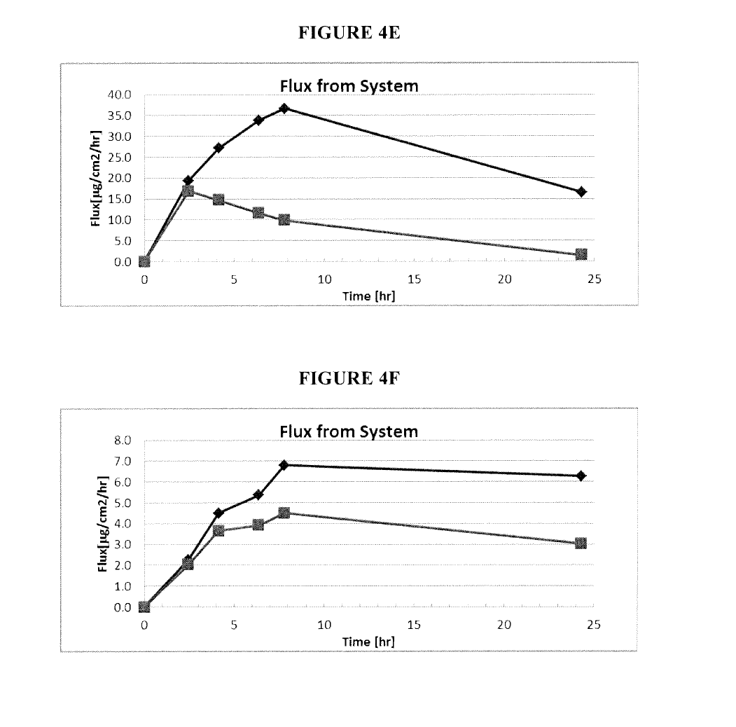

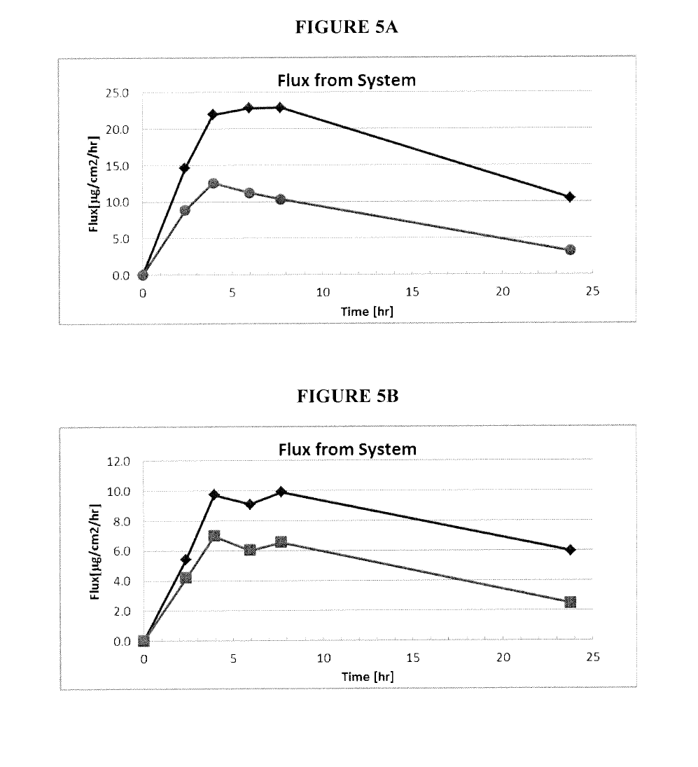

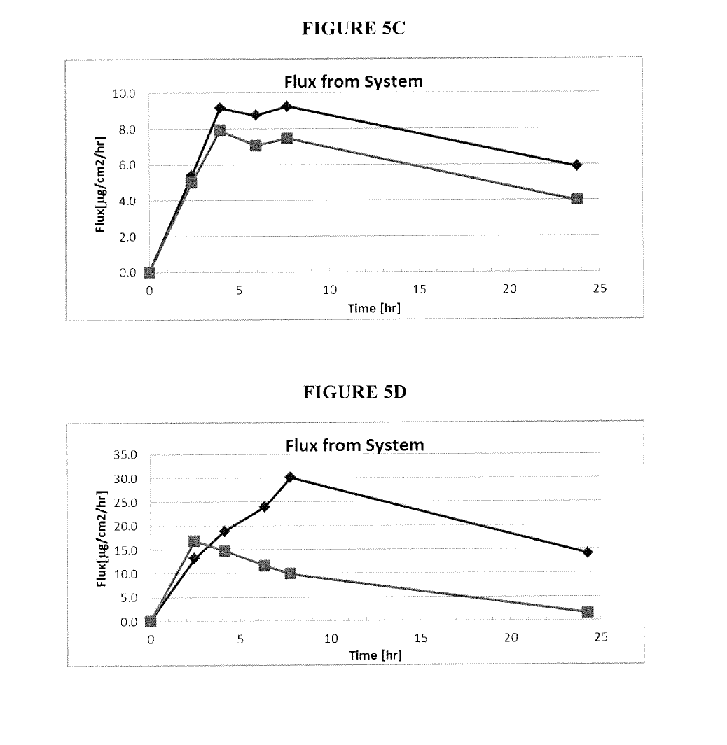

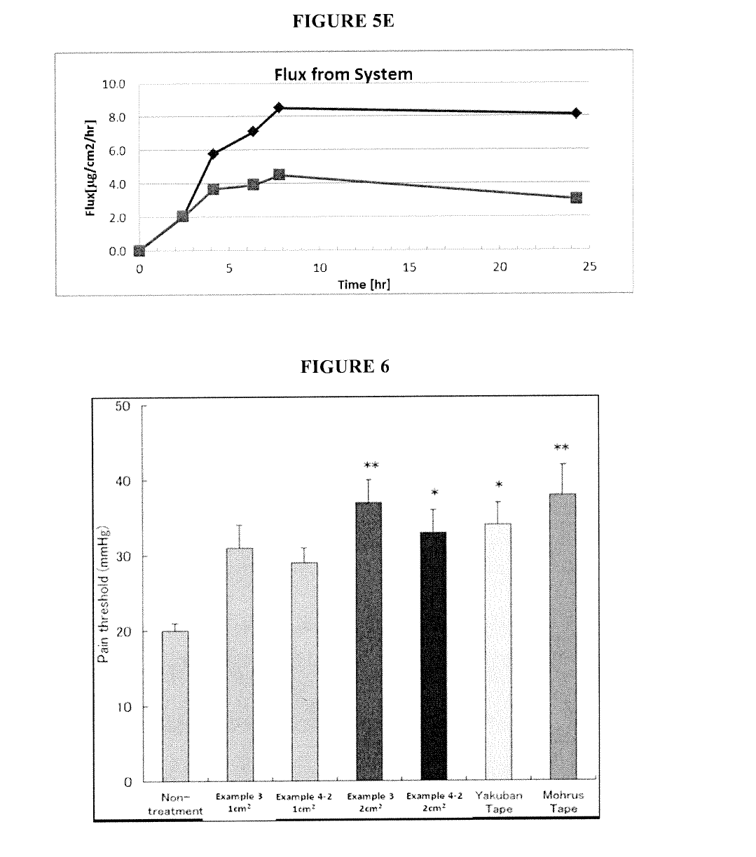

Results are shown in FIG. 4A-F (formulation 5-1) and 5A-E (formulation 5-2), and demonstrate that the polymer matrices as described herein achieve a greater, sustained drug flux than the commercial products.

### EXAMPLE 6

Systems prepared using the formulations described above were use in an animal model for pain, the yeast-induced hyperalgesia model in rats. The systems (1 cm² or 2 cm²) were topically applied to the right hind paw of the animal for 3 hours before the yeast injection. Pain threshold was measured at 3 hours after the yeast injection. Results are shown in FIG. 6. Each value represents the mean±S.E. of 10 animals. Flurbiprofen commercial patch (Yakuban Tape) and Ketprofen commercial patch (Mohrus Tape) were also evaluated as references. The formulations described herein showed potent analgesic effect, equivalent to the commercial products.

What is claimed is:

1. A composition for the transdermal delivery of an NSAID in the form of a flexible finite system for topical application, comprising a polymer matrix comprising (i) a therapeutically effective amount of an NSAID; (ii) a silicone polymer; and (iii) an acrylic polymer or an acrylic block copolymer.

2. The composition of claim 1, wherein the polymer matrix further comprises a styrene-isoprene-styrene block copolymer.

3. The composition of claim 1, wherein the polymer matrix further comprises a penetration enhancer.

4. The composition of claim 1, wherein the NSAID comprises flurbiprofen.

5. The composition of claim 1, wherein the polymer matrix comprises a non-functional acrylic polymer.

6. The composition of claim 1, wherein the polymer matrix comprises an acrylic block copolymer.

7. The composition of claim 1, wherein the polymer matrix comprises about 3% to 5% w/w flurbiprofen.

8. The composition of claim 1, wherein the polymer matrix comprises about 4 to 5% w/w acrylic polymer.

9. The composition of claim 1, wherein the polymer matrix comprises about 5% w/w acrylic block copolymer.

10. The composition of claim 1, wherein the polymer matrix comprises up to about 1% w/w styrene-isoprene-styrene block copolymer.

11. The composition of claim wherein the polymer matrix comprises at least about 80% w/w silicone polymer.

12. The composition of claim 1, wherein the system achieves delivery of the NSAID over a period of time of at least 8 hours.

13. The composition of claim 1, wherein the system achieves delivery of the NSAID over a period of time of at least 12 hours.

14. The composition of claim 1, wherein the system achieves delivery of the NSAID over a period of time of at least 24 hours.

15. The composition of claim 1, further comprising a backing layer.

16. The composition of claim 1, further comprising a flexible, occlusive backing layer.

17. The composition of claim 16, wherein the flexible, occlusive backing layer is stretchable.

18. The composition of any claim 16, wherein the flexible, occlusive backing layer is comprised of a fabric backing material coated with an occlusive coating.

19. The composition of claim 18, wherein the occlusive coating comprises polyisobutylene and/or styrene-isoprene-styrene (SIS) block copolymers.

20. A method for the transdermal delivery of an NSAID, comprising topically applying a composition as claimed in claim 1 to the skin or mucosa of a subject in need thereof.

21. A flexible, finite system for the transdermal delivery of an active agent comprising, (i) a polymer matrix comprising the active agent and one or more polymers and (ii) a flexible, occlusive backing layer comprised of a fabric backing material coated with an occlusive coating.

22. The flexible, finite system of claim 21, wherein the flexible, occlusive backing layer is stretchable.

23. The flexible, finite system of claim 22, wherein the flexible, finite system is stretchable.

24. A method for the transdermal delivery of an NSAID, comprising topically applying a flexible, finite system as claimed in claim 21 to the skin or mucosa of a subject in need thereof.
